(19) United States
(12) Patent Application Publication
(10) Pub. No.: US 2012/0296235 A1
(43) Pub. Date: Nov. 22, 2012

(54) AUTOMATED SYSTEM AND METHOD FOR PERFORMING AND MONITORING PHYSICAL THERAPY EXERCISES

(76) Inventors: Keith W. Rupp, Glendale, AZ (US); Sue Dahl-Popolizio, Peoria, AZ (US); David Bahr, Chandler, AZ (US); Jamil Loman, Phoenix, AZ (US)

(21) Appl. No.: 13/434,793

(22) Filed: Mar. 29, 2012

Related U.S. Application Data
(60) Provisional application No. 61/468,980, filed on Mar. 29, 2011.

(51) Int. Cl. A61B 5/103

(52) U.S. Cl. 600/595

(57) ABSTRACT

An automated system and method for performing and monitoring physical therapy exercises that includes the computerized motion capture of a user performing physical therapy exercises, comparison of the user's exercises to an idealized stored digital representation of the same exercises based on body type, and real time feedback to the user to enable the user to correct and complete the exercises. The system and method may also include the automated transmission of the user's physical therapy exercise session and related information to the user's healthcare professional.

### CROSS-REFERENCE TO RELATED APPLICATIONS

This application claims priority to provisional patent application having Ser. No. 61/468,980, filed Mar. 29, 2011, which is herein incorporated by reference in its entirety.

### FIELD OF INVENTION

The present invention is generally directed to an automated system and method for performing and monitoring physical therapy exercises. More particularly, the present invention is directed to an automated system and method for performing and monitoring physical therapy exercises that includes the computerized capture of a user performing physical therapy exercises, comparison of the user's exercises to an idealized stored digital representation of the same exercises based on body type, and real time feedback to the user to enable the user to correct and complete the exercises. The system and method may also include the automated transmission of the user's physical therapy exercise session and related information and results to the user's healthcare professional.

### BACKGROUND OF THE INVENTION

Physical therapy exercises are designed to address movement dysfunction. Correct performance of the exercise requires precise alignment and speed relative to other skeletal segments to be maintained. Faulty alignment or speed requires corrective feedback to achieve the ideal motion for carrying out the physical therapy exercise. Failure to maintain the ideal alignment and speed during the exercise motion can slow the process of healing, or worse, do further damage to the person performing the exercise.

Physical therapy exercises are taught and observed by trained health professionals. However, a healthcare professional's time is very costly, and some physical therapy exercises must be performed on a daily or even hourly basis to be effective. Therefore, there is a need for an accurate and cost effective way for patients or users to carry out the physical therapy exercises prescribed by their healthcare professionals and for healthcare professionals to provide feedback and further instructions to their patients/users without the need for multiple office visits in order for the patient/user to obtain optimal healing.

The present invention provides for the demonstration of a professionally-defined physical therapy exercise that is digitally captured and stored in a computerized system. An idealized exercise template can then be prescribed to the patient/user and transferred to another computing system. The present invention also provides real-time monitoring of the patient/user and thereby acts as a proxy for observation by a healthcare professional. The results of the real-time monitoring can be summarized and conveyed to the healthcare professional who can then apply appropriate modifications and corrections.

### SUMMARY OF THE INVENTION

The present invention provides an automated system and method for performing and monitoring physical therapy. First, an idealized or accurate performance of a physical exercise is captured and digitally stored as a control template for later retrieval. Several various criteria/physical characteristics about the individual performing the accurate performance of the physical exercise, such as, e.g., sex, height, weight, and body type, are stored as part of the control template. A healthcare professional then prescribes a predefined set of physical therapy exercises or physical therapy exercise set along with the number of repetitions and the number of times a day the exercises should be performed. The user then enters certain physical characteristics about themselves and selects their prescribed set of physical therapy exercises. The stored templates are then accessed and the user's physical characteristics are matched with one or more of the stored control templates.

A computerized system with motion detection cameras and/or apparatus utilizes the stored control template(s) that have been matched with the user. The user begins the prescribed physical therapy exercise and the computerized system receives data reflecting the user's movement from an attached motion-capture apparatus. The system compares the user's movements to the control template(s) that were matched to the user and determines whether the user performed one or more physical therapy exercises correctly along with the number of repetitions performed.

The system may display a silhouette or video of a body similar to the user's body shape and size performing the exercise on a nearby display device to allow the user to preview the exercise and/or to further prompt the user during performance of the exercise. The silhouette or video may show how to perform the exercise correctly, eventually prompting the user to follow along. If the user deviates from the stored control template for the exercise, immediate feedback is given in the form of audio, visual, and/or haptic feedback (such as on the motion detection device in the form of a sense of touch if the motion detection device is held by or on a user). The feedback prompts the user to perform the exercise closer to the exercise stored on the control template.

The system continues to monitor the user over the arc of the exercise, noting when each exercise is completed. If the exercise prescription involves a certain range of complete repetitions, the user may be given feedback on this as well. After the session is completed, the system stores which exercises the user has attempted, how accurately they were performed, and how many were completed. All of this information can then be summarized to the user and/or transmitted to the user's healthcare professional.

In one exemplary embodiment, the automated system for performing and monitoring physical therapy includes a database containing a plurality of stored physical therapy exercise templates (or control templates) based on a human body's physical characteristics, one or more sensors or a motion capture apparatus for sensing the physical movements of a user, a processor for receiving the physical movements of the user sensed by the motion capture apparatus and comparing the physical movements of the user to at least one of the stored physical therapy exercise templates, and a display device for providing real-time feedback to the user to indicate correction and/or completion of a physical therapy exercise relating to the physical therapy exercise template. The automated system of the present invention may also include a database for storing the information relating to the user's physical therapy exercise correction and/or completion. In addition, the automated system of the present invention may include an input digitizer coupled or connected to the processor for digitizing the data from the motion capture apparatus.

The automated system of the present invention may further include a network for transmitting the information relating to the user's physical therapy exercise correction and/or completion to a user's healthcare professional. The system may also include a database for storing modifications, corrections, and instructions provided by the user's healthcare professional in response to reviewing the information relating to the user's physical therapy exercise correction and/or completion which can then be accessed by the user.

In one aspect of the invention, the plurality of stored physical therapy exercise templates (or control templates) may be based on one or both of a) a human body's sex, weight, and height and b) a body type of a human body. In another aspect of the invention, if the physical therapy exercise templates are based on a body type of a human body, they may be based on one or more of an ectomorph body type, a mesomorph body type, an endomorph body type, or a combination thereof.

Further, with respect to the display device for providing real-time feedback to the user, the display device may provide a display of a human body or simulated human body similar to the user's body that is performing the physical therapy exercise where the display of the human body or simulated human body shows a correct performance of the physical therapy exercise. The display device may further display a percentage of accuracy of the user's performance of the physical therapy exercise as compared to the correct performance of the physical therapy exercise provided by the stored control template. In addition, the display device may provide real-time feedback information relating to one or more of a user's direction of movement, the user's velocity of movement, or the user's relative position of bone joints as compared to the correct performance of the physical therapy exercise provided by the stored control template. The display device may also provide audible real-time feedback. Further, real-time feedback may be provided by having a portion of the motion capture apparatus provide audible and/or haptic feedback.

In another exemplary embodiment of the invention, the automated system for performing and monitoring physical therapy includes a host server, a database containing a plurality of stored physical therapy exercise templates (or control templates) based on a human body's physical characteristics, a motion capture apparatus in communication with the host server via a network for sensing the physical movements of a user, a processor for receiving the physical movements of the user and comparing the physical movements of the user to at least one of the stored physical therapy exercise templates (control templates), and a display device with user interface for inputting user information and providing real-time feedback to the user to indicate correction and/or completion of a physical therapy exercise relating to the physical therapy exercise template. The automated system may also provide for real-time haptic feedback which is felt through a portion of the motion capture apparatus if the user is holding or wearing a portion of the capture apparatus. This embodiment of the automated system may also include a database for storing the information relating to the user's physical therapy exercise correction and/or completion. This embodiment of the automated system may also include a database, which can be accessed by the user, for storing modifications, corrections, and/or instructions provided by the user's healthcare professional in response to receiving and reviewing the information relating to the user's physical therapy exercise correction and/or completion via the network.

In one aspect of this exemplary embodiment, the plurality of stored physical therapy exercise templates (or control templates) may be based on one or both of a) a human body's sex, weight, and height and b) a body type of a human body. In another aspect of this exemplary embodiment, if the physical therapy exercise templates are based on a body type of a human body, they may be based on one or more of an ectomorph body type, a mesomorph body type, an endomorph body type, or a combination thereof.

Further, with respect to the display device for providing real-time feedback to the user, the display device in this exemplary embodiment may provide a display of a human body or simulated human body similar to the user's body that is performing the physical therapy exercise where the display of the human body or simulated human body shows a correct performance of the physical therapy exercise. The display device of this exemplary embodiment may further display a percentage of accuracy of the user's performance of the physical therapy exercise as compared to the correct performance of the physical therapy exercise provided by the stored control template. In addition, the display device of this exemplary embodiment may provide real-time feedback information relating to one or more of a user's direction of movement, the user's velocity of movement, or the user's relative position of bone joints as compared to the correct performance of the physical therapy exercise provided by the stored control template. The display device of this exemplary embodiment may also provide audible real-time feedback. Further, real-time feedback may be provided by having a portion of the motion capture apparatus of this exemplary embodiment provide audible and/or haptic feedback.

The present invention is also directed to an automated method for performing and monitoring physical therapy which, in one exemplary embodiment, includes a) creating and storing a plurality of control templates relating to accurate physical movements for a plurality of physical therapy, exercises, b) inputting information relating to a user's physical body characteristics, c) selecting a prescribed physical therapy exercise set, d) matching the user's physical body characteristics with one or more of the stored control templates, e) instructing the user to perform one or more physical therapy exercises, f) detecting the user's movements with a motion capture apparatus, g) comparing the user's physical movements to the one or more control templates, h) providing real-time feedback to enable the user to correct to correct the user's physical movements to match the one or more control templates and i) storing information relating to the user's performance and completion of the one or more physical therapy exercises. The automated method may also include the steps of j) transmitting the stored information relating to the user's performance and completion of the one or more physical exercises to the user's healthcare professional, k) storing feedback and/or further instruction provided by the user's healthcare professional to the user, l) transmitting the feedback and/or further instruction provided by the healthcare professional to the user, and m) repeating steps b through l until the user has completed his or her physical therapy protocol.

The automated method may further include the step of presenting one or more of the stored control templates to the user for viewing prior to instructing the user to perform the physical therapy exercises or for prompting the user to follow the accurate physical movements for performing the physical therapy exercises. In one aspect of the automated method, the step of creating and storing a plurality of control templates may include the steps of: a) entering an individual's physical data relating to the user's sex, weight, and height, b) capturing the individual's accurate performance of a physical therapy exercise by capturing one or more types of data relating to the performance, c) digitizing, processing, and normalizing the capture data, d) storing the processed capture data and the individual's physical data characteristics as a control template; and e) repeating steps a through d for a plurality of different individuals. In another aspect of the automated method, the step of creating and storing a plurality of control templates may include the steps of: a) capturing an accurate performance of a physical therapy exercise carried out by an individual having an ectomorph body type, an individual having a mesomorph body type, and an individual having an endomorph body type by capturing one or more types of data relating to each of the performances, b) digitizing, processing, and normalizing the capture data, c) storing the processed capture data and the individual's body type as control templates, d) repeating steps a through c for a plurality of individuals to determine if they have a unique bodytype in comparison to the body types already stored as control templates, and e) storing the processed capture data relating to any unique body types as additional control templates.

### BRIEF DESCRIPTION OF THE DRAWINGS

The figures illustrate various embodiments of the present invention by way of example, and not by way of limitation. Embodiments of the present invention may include part or all of the features shown in one of these figures, or may include features from two or more figures. Embodiments of the present invention may also include features described in the specification, or elements of features described in the specification. Furthermore, embodiments of the present invention may include features that would be familiar to a person of ordinary skill in the art having studied this document. Thus, a more complete understanding of the present invention may be derived by referring to the detailed description and claims when considered in connection with the drawing figures. Where like reference numbers refer to similar elements throughout the figures, and

FIG. 1 is a block diagram of an exemplary embodiment of an automated system for performing and monitoring physical therapy exercises;

FIG. 2 is a flowchart of an exemplary embodiment of an automated method for performing and monitoring physical therapy exercises;

FIG. 3 is a flowchart of an exemplary embodiment of the steps which may comprise the step of creating and storing a plurality of control templates also known as the capture method for creating control templates;

FIG. 4 is a flowchart of another exemplary embodiment of the automated method for performing and monitoring physical therapy exercise also know as the user method for performing and monitoring physical therapy exercise;

FIG. 5 is a schematic showing components of the automated system for performing and monitoring physical therapy exercises in use by a user; and

FIG. 6 is a block diagram of another exemplary embodiment of an automated system for performing and monitoring physical therapy exercises.

### DETAILED DESCRIPTION

The detailed description of exemplary embodiments herein makes reference to the accompanying drawings and pictures, which show the exemplary embodiment by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in a different order and are not limited to the order presented. Moreover, any of the functions or steps may be outsourced to or performed by one or more third parties. Furthermore, any reference to singular includes plural embodiments, and any reference to more than one component may include a singular embodiment.

For the sake of brevity, conventional data networking, application development and other functional aspects of the systems (and components of the individual operating components of the systems) may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

The various system components discussed herein may include one or more of the following: a host server or other computing systems including a processor for processing digital data; a memory coupled to the processor for storing digital data; a motion capture apparatus coupled to the processor for capturing data relating to a user's physical motions, an input digitizer coupled to the processor and the motion capture apparatus for inputting digital data relating to the capture data; an application program stored in the memory and accessible by the processor for directing processing of digital data by the processor; a display device coupled to the processor for displaying information derived from digital data processed by the processor; and a plurality of databases. Various databases used herein may include, but are not limited to, patient or client data, physical therapy exercise control templates, data and information relating to a user's physical therapy exercise correction and/or completion relating to the control templates, modifications, corrections, and/or instructions from healthcare providers based on receipt and review of user exercise correction and/or completion, etc. As those skilled in the art will appreciate, user computer may include an operating system as well as various conventional support software and drivers typically associated with computers. The computer may include any suitable personal computer, network computer, workstation, minicomputer, mainframe or the like. The user's computer can be in a home or business environment with access to a network. In an exemplary embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, online communications, satellite communications, off-line communications, wireless communications, transponder communications, local area network (LAN), wide area network (WAN), networked or linked devices, keyboard, mouse and/or any suitable communication or data input modality. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

Any databases discussed herein may include relational, hierarchical, graphical, or object-oriented structure and/or any other database configurations. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked. In accordance with one embodiment, any suitable data storage technique may be utilized to store data without a standard format. Data sets may be stored using any suitable technique, including, for example, storing individual files; implementing a domain whereby a dedicated file is selected that exposes one or more elementary files containing one or more data sets; using data sets stored in individual files using a hierarchical filing system; data sets stored as records in a single file; Binary Large Object (BLOB); stored as ungrouped data elements encoded using various data elements; and/or other proprietary techniques that may include fractal compression methods, image compression methods, etc.

In one exemplary embodiment, the ability to store a wide variety of information in different formats is facilitated by storing the information as a BLOB. Thus, any binary information can be stored in a storage space associated with a data set. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data associated with physical therapy exercise control templates, the physical movements of multiple unrelated users captured from motion capture apparatus, and data relating to the comparison of physical movements of a user with one or more control templates. For example, a first data set which may be stored may be provided by a first party, a second data set which may be stored may be provided by an unrelated second party, and yet a third data set which may be stored, may be provided by a third party unrelated to the first and second party. Each of these three exemplary data sets may contain different information that is stored using different data storage formats and/or techniques. Further, each data set may contain subsets of data that also may be distinct from other subsets.

As stated above, in various embodiments, the data can be stored without regard to a common format. However, in one exemplary embodiment, the data set (e.g., BLOB) may be annotated in a standard manner when provided for comparing the data to control templates to produce real-time feedback. The annotation may comprise a short header, trailer, or other appropriate indicator related to each data set that is configured to convey information useful in managing the various data sets. For example, the annotation may be called a “condition header”, “header”, “trailer”, or “status”, herein, and may comprise an indication of the status of the data set or may include an identifier correlated to a specific owner of the data.

The data set annotation may also be used for other types of status information as well as various other purposes. For example, the data set annotation may include security information establishing access levels. The access levels may, for example, be configured to permit only certain individuals, users, healthcare professionals, companies, or other entities to access data sets, or to permit access to specific data sets based on owner of the data. Furthermore, the security information may restrict/permit only certain actions such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Participation originating at a web user/patient may pass through a firewall in order to prevent unauthorized access from users of other networks.

The computers discussed herein may provide a suitable website or other internet-based graphical user interface which is accessible by users to obtain information needed to carry out the automated system via access through the interne. Alternatively, the automated system described herein may comprise two separate computerized systems—one for capturing data including motion related data for creating a plurality of control templates for correctly carrying out physical therapy exercises which can then be downloaded to a second computerized system utilized by a user which also includes the capture of motion related data for comparison to the control templates.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), helper applications, plug-ins, and the like. A server may include a web service that receives a request from a web server, the request including a URL and an IP address. The web server retrieves the appropriate web pages and sends the data or applications for the web pages to the IP address. Web services are applications that are capable of interacting with other applications over a communications means, such as the internet.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page or user input page of a computer program such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language with the various algorithms being implemented with any combination of data structures, objects, processes, routines or other programming elements. Further, it should be noted that the system may employ any number of conventional techniques for data transmission, signaling, data processing, network control, and the like.

As used herein, the term “user”, “patient”, or “participant”, may be used interchangeably with each other. Each user is equipped with a computing device having input capabilities, a display device (which may include the input capabilities), and a motion capture apparatus in order to interact with and utilize the automated system online via a network. Alternatively, each user is equipped with a computing system having input capabilities, a display device, and a motion capture device which are all coupled to one another so that the automated system can be used by downloading a computer program containing control templates to the computing system.

As will be appreciated by one of ordinary skill in the art, the system may be embodied as a customization of an existing system, an add-on product, upgraded software, a stand alone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the, block diagrams and flowchart illustrations, respectively, can be implemented by computer program instructions.

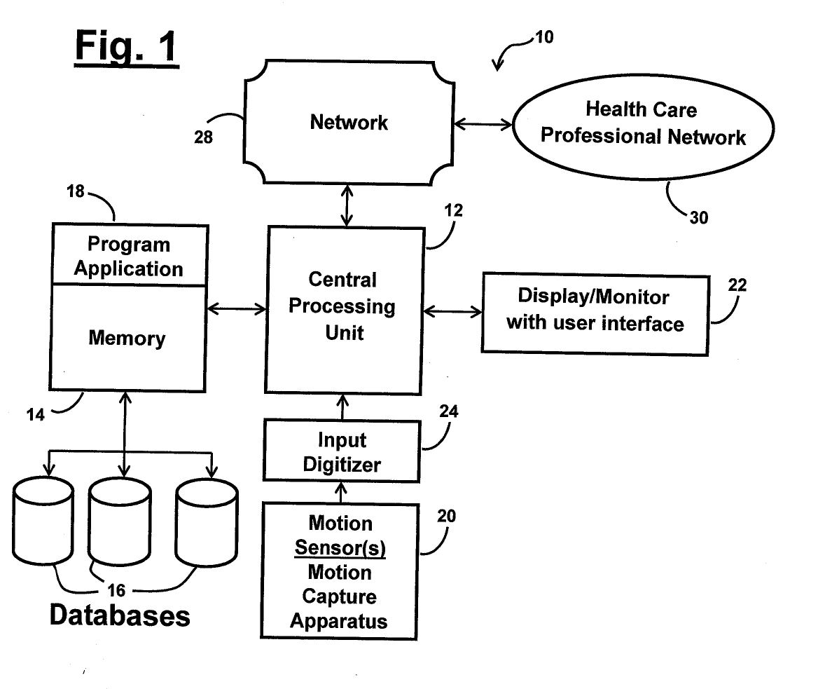

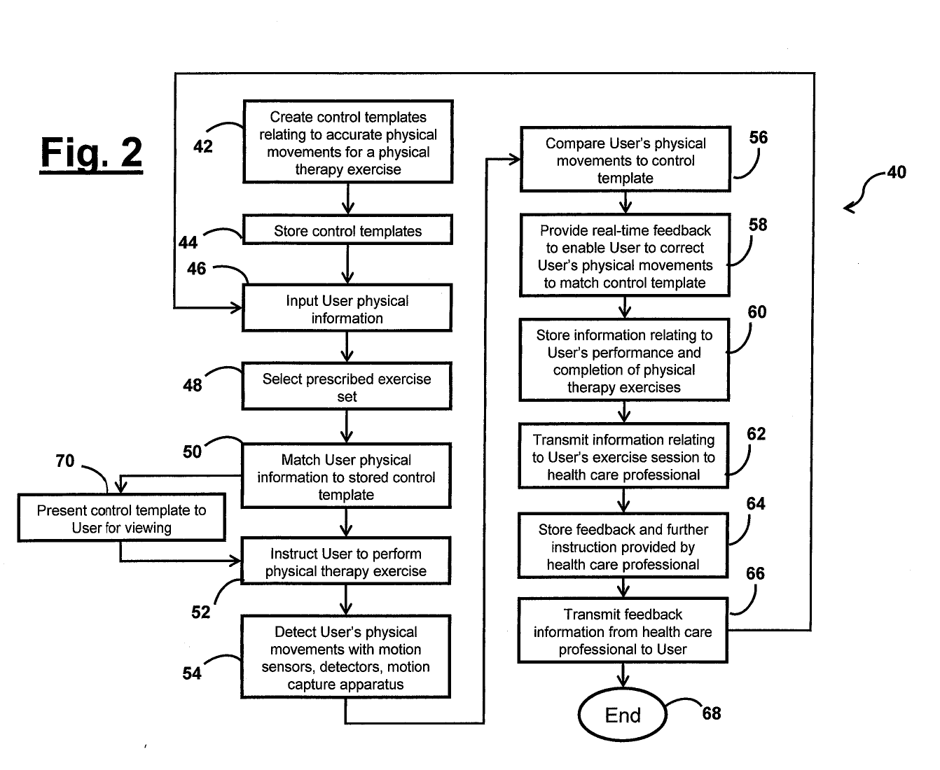

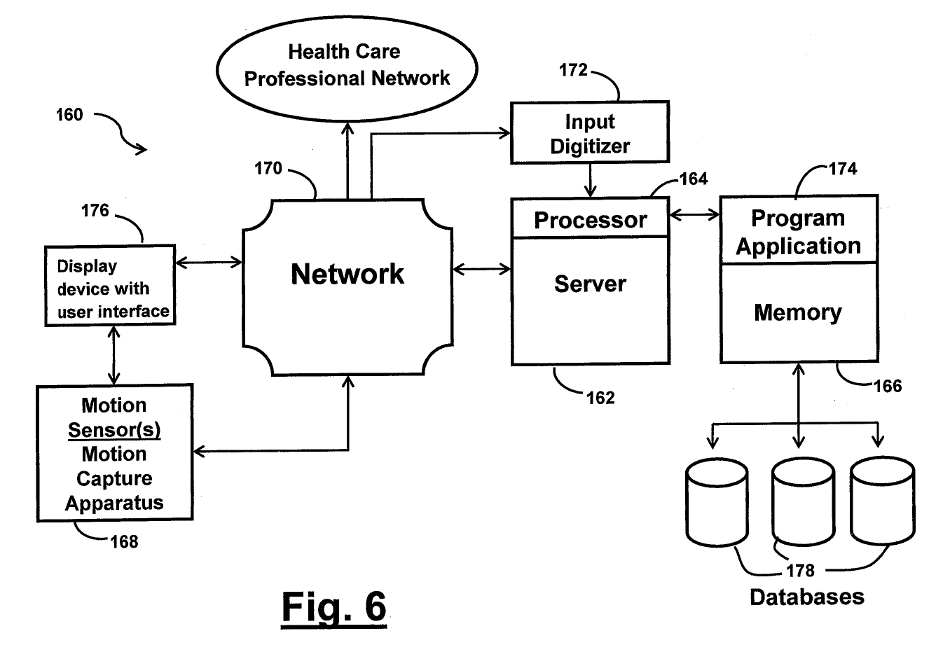

Referring now to FIGS. 2-5, the process flows, apparatus schematics, and screenshots depicted are merely embodiments and are not intended to limit the scope of the invention as described herein. For example, the steps recited in any of the method or process descriptions may be executed in different orders and are not limited to the order presented. It will be appreciated that the following description makes appropriate references not only to the steps and user interface elements depicted in FIGS. 2-5, but also to the various system components as described with reference to FIGS. 1 and 6.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user windows, webpages, websites, web forms, prompts, etc. Practitioners will appreciate that the illustrated steps described herein may comprise in any number of configurations including the use of windows, webpages, web forms, popup windows, prompts and the like. It should be further appreciated that the multiple steps as illustrated and described may be combined into single webpages and/or windows but have been expanded for the sake of simplicity. In other cases, steps illustrated and described as single process steps may be separated into multiple webpages and/or windows but have been combined for simplicity.

Benefits, other advantages, and solutions to problems have been described herein with regard to specific embodiments. However, the benefits, advantages, solutions to problems, and any element(s) that may cause any benefit, advantage, or solution to occur or become more pronounced are not to be construed as critical, required, or essential features or elements of any or all the claims or the invention. The scope of the invention is accordingly to be limited by nothing other than the appended claims, in which reference to an element in the singular is not intended to mean “one and only one” unless explicitly so stated, but rather “one or more.” All structural and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “critical”.

Turning now to the Figures, FIG. 1 shows a block diagram of an exemplary embodiment of an automated system 10 for performing and monitoring physical therapy exercises in accordance with the present invention. System 10 includes a processor 12 having a memory 14 for storing databases 16 and a program application 18 for carrying out one or more embodiments of the automated method of the present invention for performing and monitoring physical therapy exercises. System 10 also includes a motion capture apparatus 20 coupled to the processor 12 for capturing data relating to a user's physical motions and a display device 22 coupled to the processor for providing real-time feedback to a user. An input digitizer 24 may be coupled to the processor 12 and the motion capture apparatus 20 for inputting digital data relating to the capture data. The system also includes a user interface (not shown) coupled to the processor for inputting information relating to a user's physical body characteristics. The processor 12 can be connected to a network 28, such as, for example, the internet, an intranet, or any other type of network, so that data stored in databases 16 relating to a user's performance and/or completion of prescribed physical therapy exercises may be transmitted to a healthcare provider or healthcare professional network 30 and a user can then receive feedback and/or further instruction from a healthcare provider or healthcare professional network in response to a user's performance and completion of prescribed physical therapy exercises.

The motion capture apparatus 20 may comprise any number of motion detection devices and/or motion sensing devices that may include, but are not limited to, sensors for detecting radio frequency identification, cameras, gyroscopes, accelerometers, global positioning system components, light and range detectors, etc. Motion capture may be performed by devices, components, and/or sensors that are independent from the user for whom motion is being detected or motion capture may be performed by sensors, components or other devices that are held by, worn by, or attached to a user.

Memory 14 may comprise any collection and arrangement of volatile or non-volatile, local or remote devices suitable for storing data such as, for example, random access memory (RAM) devices, read only memory (ROM) devices, magnetic storage devices, or other suitable data storage devices. Display device/display monitor 22 may include, but is not limited to, computer monitors, television sets, light-emitting diodes (LEDs), and liquid crystal displays (LCDs).

FIG. 2 is a is a flowchart of an exemplary embodiment of an automated method 40 for performing and monitoring physical therapy exercises and prescribed physical therapy protocols. Automated method 40 includes the steps of creating control templates relating to accurate physical movements for a physical therapy exercise in step 42, storing the control templates in a database in step 44, inputting information relating to a user's physical body characteristics in step 46, selecting a prescribed physical therapy exercise set in step 48, matching the user's physical body characteristic information to one or more stored control templates in step 50, instructing the user to perform one or more physical therapy exercises in step 52, detecting the user's physical movements with a motion capture apparatus in step 54, comparing the user's physical movements to one or more of the control templates in step 56, providing real-time feedback to enable a user to correct the user's physical movements to match the control template(s) in step 58, and storing the information relating to the user's performance and completion of the one or more physical therapy exercises in step 60. The automated method may further include the steps of transmitting the stored information relating to the user's exercise session (which may include, but is not limited to, performance and completion information relating to the user's physical therapy exercises) to a healthcare professional in step 62, storing feedback and/or further instruction provided by the healthcare professional to the user in step 64, transmitting the feedback and/or further instruction from the healthcare professional to the user in step 66, and repeating steps 46 through 66 until the user has completed his/her physical therapy prescription and ends the automated method 40 in step 68.

The automated method 40 may also include the step of presenting one or more control templates to the user for viewing in step 70 prior to step 52 which instructs the user to perform the physical therapy exercise.

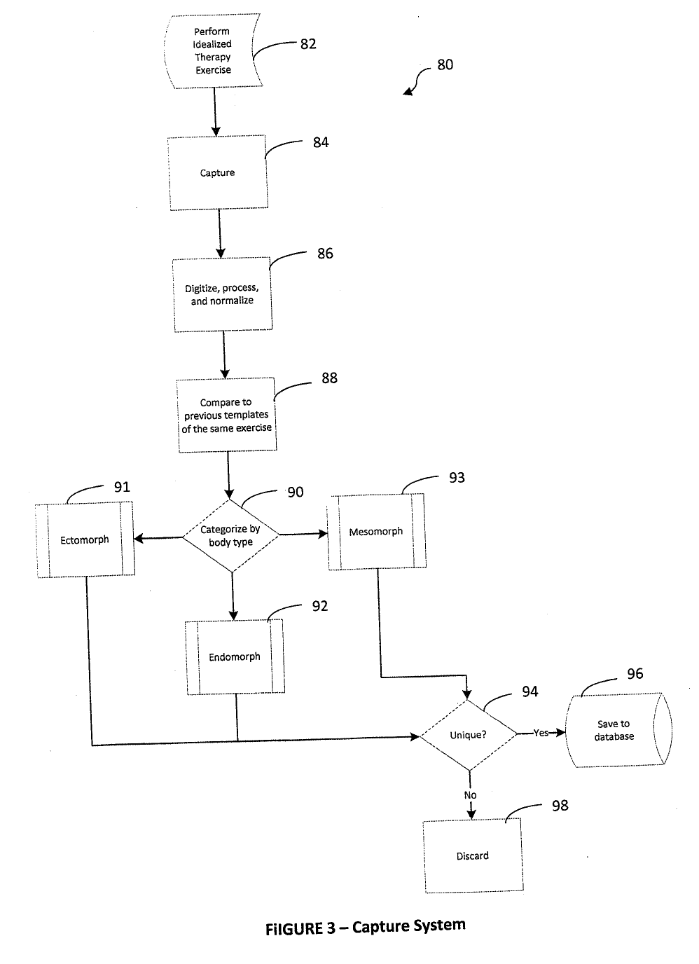

FIG. 3 is a flowchart of an exemplary embodiment of the steps which may comprise the step of creating and storing a plurality of control templates also known as the capture method 80 for creating control templates. Capture method 80 includes the steps of performing an idealized therapy exercise in step 82, capturing the physical movements of the idealized therapy exercise in step 84 by using a motion capture apparatus, digitizing, processing, and normalizing the capture data in step 86, comparing the processed capture data to previous control templates of the same therapy exercise after creation of initial control templates in step 88, categorize the processed data by body type in step 90 where the categorized body type is one of an ectomorph body type. 91, and endomorph body type 92, and a mesomorph body type 93, determine whether the processed date is unique in step 94 with respect to the existing body type control templates for which it was categorized in step 90, save to the database as a new control template in step 96 if it is unique, and discard in step 98 if it is not unique.

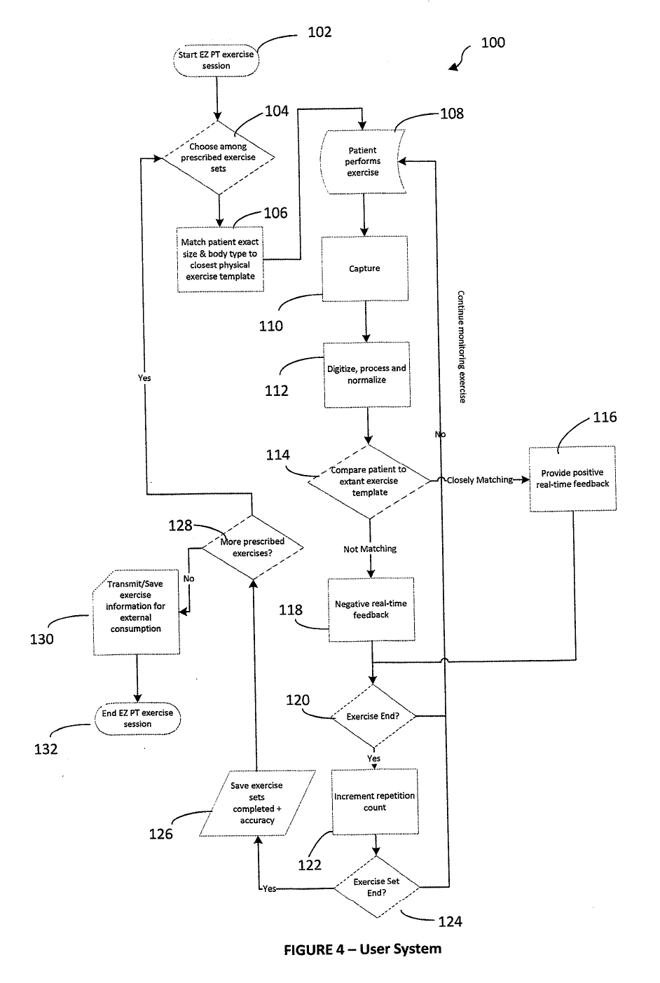

A flowchart of another exemplary embodiment of the automated method for performing and monitoring physical therapy exercise also know as the user method 100 for performing and monitoring physical therapy exercise is shown in FIG. 4. User method 100 includes the steps of starting the automated physical therapy exercise session in step 102, choosing among a prescribed exercise set in step 104, matching a user's size and body type with a physical exercise control template in step 106, having the patient perform the physical exercise in step 108, capturing the user's physical movements while performing the physical exercise in step 110 with a motion capture device, digitizing, processing, and normalizing the capture data in step 112, comparing the user's physical movements to the previously matched physical exercise control template in step 114, providing positive real-time feedback in step 116 if the user's movements closely match the control template and providing negative real-time feedback in step 118 if the user's movements do not closely match the control template, determine of the physical exercise has ended in step 120 and, if it has, perform increment repetition counts in step 122, determine if the exercise set has ended in step 124 and, if it has, save information relating to the completion and accuracy of performing the exercise sets in step 126, determine if there are more prescribed exercise to complete in step 128, and, if there are more to complete return to step 104 to choose among a prescribed exercise set. If there are no more exercise sets to complete, transmit and/or save the information relating to the physical therapy exercise session in step 130 and end the physical therapy exercise session in step 132.

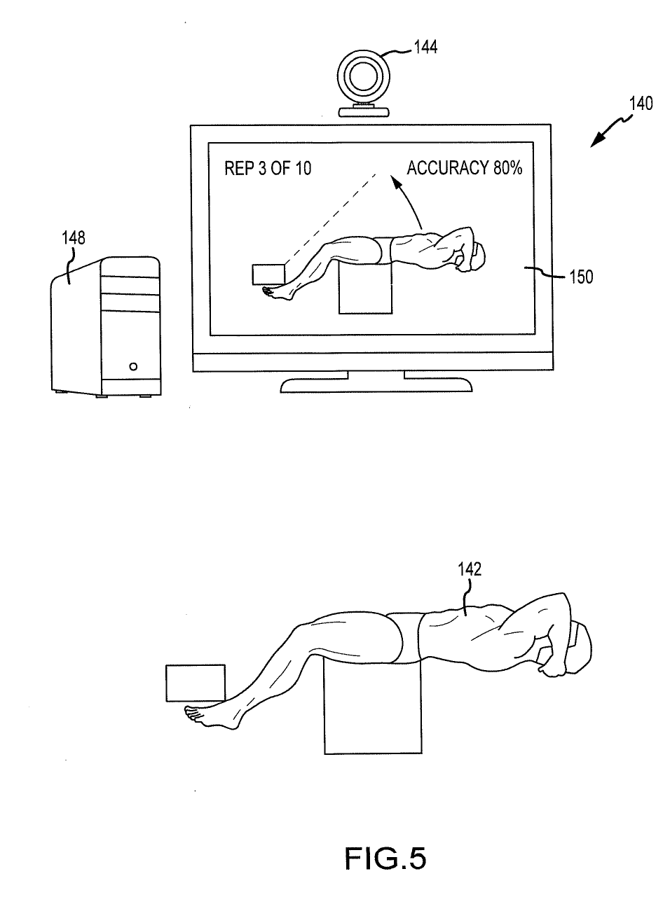

FIG. 5 is a schematic 140 showing components of the automated system for performing and monitoring physical therapy exercises in use by a user. Schematic 140 shows a user 142 performing a prescribed exercise, a motion capture device 144 capturing the physical movements of the user performing the exercise, a processor 148 which is comparing the user's physical movements to a matched control template for the exercise that the user is performing, and a display device 150 which is displaying real-time feedback to the user so that the user can determine his accuracy in performing the exercise and correct his movements if necessary. The display device is also showing the percent accuracy of the user's movement as compared to the matched control template, and the number of repetitions that the user has completed in the set of repetitions that the user needs to complete.

FIG. 6 is a block diagram of another exemplary embodiment of an automated system 160 for performing and monitoring physical therapy exercises. System 160 includes a host server 162 which includes a processor 164 for processing digital data; a memory 166 coupled to the processor for storing digital data; a motion capture apparatus 168 coupled to the processor via a network 170 for capturing data relating to a user's physical motions, an input digitizer 172 coupled to the processor 164 for inputting digital data via the network relating to the capture data obtained from the motion capture device; an application program 174 stored in the memory and 166 accessible by the processor 164 for directing processing of digital data by the processor 164; a display device 176 coupled to the processor 164 via the network for displaying information derived from digital data processed by the processor 164; and a plurality of databases 178. Various databases used herein may include, but are not limited to, patient or client data, physical therapy exercise control templates, data and information relating to a user's physical therapy exercise correction and/or completion relating to the control templates, modifications, corrections, and/or instructions from healthcare providers based on receipt and review of user exercise correction and/or completion, etc. The system 160 also includes connection to a healthcare professional network 180 so that data from the databases relating to a user's completion and performance of a prescribed physical therapy protocol can be automatically transmitted to the healthcare professional network upon completion of the physical therapy protocol. System 160 is designed to be used by accessing a network such as the internet as long as the user accessing the internet has a motion capture apparatus and display device that are able to be connected to the internet for capture and feedback steps.

The systems and methods of the present invention can provide automatic follow through of all steps so that a user and the user's healthcare professional are automatically provided with information (such as real-time feedback, data and information relating to a user's physical therapy exercise correction and/or completion relating to the control templates, and modifications, corrections, and/or instructions from healthcare providers based on receipt and review of user exercise correction and/or completion, etc.) so that a user can cost effectively and efficiently receive the intended results of the physical therapy (such as healing) without having to make time consuming and costly visits to the offices of a healthcare professional.

The detailed description of exemplary embodiments of the invention herein shows various exemplary embodiments and the best modes, known to the inventor at this time, of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included figures are non-limiting illustrations of the exemplary embodiments and modes, which similarly avail themselves to any equivalent modes or embodiments that are known or obvious to those reasonably skilled in the art.

Unless specifically noted, it is the Applicant's intent that the words and phrases in the specification and the claims be given the commonly accepted generic meaning or an ordinary and accustomed meaning used by those of ordinary skill in the applicable arts. In the instance where these meanings differ, the words and phrases in the specification and the claims should be given the broadest possible, generic meaning. If any other special meaning is intended for any word or phrase, the specification will clearly state and define the special meaning.

1. An automated system for performing and monitoring physical therapy exercises comprising:
a database containing a plurality of stored physical therapy exercise templates based on a human body's physical characteristics;
a motion capture apparatus for sensing the physical movements of a user;
a processor for receiving the physical movements of the user sensed by the motion capture apparatus and comparing the physical movements of the user to at least one of the stored physical therapy exercise templates; and
a display device for providing real-time feedback to the user to indicate correction and/or completion of a physical therapy exercise relating to the physical therapy exercise template.

2. The automated system of claim 1 further comprising a database for storing the information relating to the user's physical therapy exercise correction and/or completion.

3. The automated system of claim 2 further comprising a network for transmitting the information relating to the user's physical therapy exercise correction and/or completion to a user's healthcare professional.

4. The automated system of claim 3 further comprising a database for storing at least one of modifications, corrections, and instructions provided by the user's healthcare professional in response to reviewing the information relating to the user's physical therapy exercise correction and/or completion which can be accessed by the user.

5. The automated system of claim 1 wherein the plurality of stored physical therapy exercise templates based on a human body's physical characteristics comprises a plurality of stored physical therapy exercise templates based on at least one of a) a human body's sex, height, and weight and b) a body type of a human body.

6. The automated system of claim 5 wherein the plurality of stored physical therapy exercise templates based on a body type of a human body comprise a plurality of physical therapy exercise templates based on one or more of an ectomorph body type, a mesomorph body type, an endomorph body type, or a combination thereof.

7. The automated system of claim 1 wherein the display device for providing real-time feedback to the user provides a display of a human body similar to a body of the user performing the physical therapy exercise which shows a correct performance of the physical therapy exercise.

8. The automated system of claim 7 wherein the display device further displays a percentage of accuracy of the user's performance of the physical therapy exercise as compared to the correct performance of the physical therapy exercise provided by the stored template.

9. The automated display device of claim 7 wherein the display device provides real-time feedback information relating to at least one of the user's direction of movement, the user's velocity of movement, and the user's relative position of bone joints as compared to the correct performance of the physical therapy exercise provided by the stored template.

10. The automated system of claim 7 wherein the display device provides audible feedback and/or a portion of the motion capture apparatus provides haptic feedback and/or audible feedback.

11. An automated system for performing and monitoring physical therapy exercise comprising:
a host server;
a database containing a plurality of stored physical therapy exercise templates based on a human body's physical characteristics;
a motion capture apparatus in communication with the host server via a network for sensing the physical movements of a user;
a processor for receiving the physical movements of the user and comparing the physical movements of the user to at least one of the stored physical therapy exercise templates; and
a display device with user interface for inputting user information and providing real-time feedback to the user to indicate correction and/or completion of a physical therapy exercise relating to the physical therapy exercise template.

12. The automated system of claim 12 further comprising a database for storing the information relating to the user's physical therapy exercise correction and/or completion.

13. The automated system of claim 13 further comprising a database, which can be accessed by the user, for storing at least one of modifications, corrections, and instructions provided by a user's healthcare professional in response to the healthcare professional receiving and reviewing the information relating to the user's physical therapy exercise correction and/or completion via the network.

14. The automated system of claim 12 wherein the plurality of stored physical therapy exercise templates based on a human body's physical characteristics comprises a plurality of stored physical therapy exercise templates based on at least one of a) a human body's sex, height, and weight and b) a body type of a human body.

15. The automated system of claim 12 wherein the display device for providing real-time feedback to the user provides a display of a human body similar to a body of the user performing the physical therapy exercise which shows a correct performance of the physical therapy exercise.

16. An automated method for performing and monitoring physical therapy comprising:
a) creating and storing a plurality of control templates relating to accurate physical movements for a plurality of physical therapy exercises;
b) inputting information relating to a user's physical body characteristics;
c) selecting a prescribed physical therapy exercise set;
d) matching the user's physical body characteristics with one or more of the stored control templates;
e) instructing the user to perform one or more physical therapy exercises;
f) detecting the user's movements with a motion capture apparatus;
g) comparing the user's physical movements to the one or more control templates;
h) providing real-time feedback to enable the user to correct to correct the user's physical movements to match the one or more control templates; and
i) storing information relating to the user's performance and completion of the one or more physical therapy exercises.

17. The automated method of claim 16 further comprising the steps of:
j) transmitting the stored information relating to the user's performance and completion of the one or more physical exercises to a healthcare professional for review and feedback;
k) storing at least one of feedback and further instruction provided by the healthcare professional;
l) transmitting the at least one of feedback and further instruction provided by the healthcare professional to the user; and
m) repeating steps b through l until the user has completed his/her physical therapy.

18. The automated method of claim 16 further comprising the step of presenting the one or more stored control templates to the user for viewing prior to instructing the user to perform one or more physical therapy exercises.

19. The automated method of claim 16 wherein the step of creating and storing a plurality of control templates comprises the steps of:
a) entering an individual's physical data relating to the user's sex, weight, and height;
b) capturing the individual's accurate performance of a physical therapy exercise by capturing one or more types of data relating to the performance;
c) digitizing, processing, and normalizing the capture data;
d) storing the processed capture data and the individual's physical data characteristics as a control template; and
e) repeating steps a through d for a plurality of different individuals.

20. The automated method of claim 16 wherein the step of creating and storing a plurality of control templates comprises the steps of:
a) capturing an accurate performance of a physical therapy exercise carried out by an individual having an ectomorph body type, an individual having an mesomorph body type, and an individual having an endomorph body type by capturing one or more types of data relating to each of the performances;
b) digitizing, processing, and normalizing the capture data;
c) storing the processed capture data and the individual's body type as control templates;
d) repeating steps a through c for a plurality of individuals to determine if they have a unique body type in comparison to the body types already stored as control templates; and
e) storing the processed capture data relating to any unique body types as additional control templates.
